(19) United States
(12) Patent Application Publication
(10) Pub. No.: US 2010/0121263 A1
(43) Pub. Date: May 13, 2010

(54) SYSTEM AND METHOD FOR ELICITING MILK FORM MAMMALS

(76) Inventors: Elona Farka, Patra (GR); Andrea Pappa, Patra (GR); Georgios Pappageorgiou, Athens (GR)

Correspondence Address: KALIKO & ASSOCIATES, L.L.C., 500 NORTH FRANKLIN TURNPIKE, RAMSEY, NJ, 07446, US

(21) Appl. No.: 12/449,054

(22) PCT Filed: Jan. 17, 2008
(86) PCT No.: PCT/GR2008/000003

§ 371 Date: Jul. 22, 2009

(30) Foreign Application Priority Data
Jan. 23, 2007 (GR) 20070100041

(51) Int. Cl. A61M 1/06; A01J 5/16; A01J 5/007

(52) U.S. Cl. 604/74; 119/14.38; 119/14.02; 119/14.38

(57) ABSTRACT

The invention provides a system and a method of eliciting milk from all mammals that possess teats, including but not limited to dairy animals and humans. A system may be comprised of an elastic system and a vacuum supply.

A method may include a milk elicitation system comprised of an elastic system and a vacuum supply. Through the application of vacuum, the elastic system forms, differentiates, seals and adapts to a teat, from the area it ends until up to where the udder ends and it begins, without coming in contact with the teat-end sphincter/lactiferous duct(s). The elastic system interacts proportionately according to the tendencies expressed by teat physiology and dynamics according to the milk pressure change, blood flow, and the effects of applied vacuum, throughout the milk elicitation process, resulting in continuous and complete milk elicitation and supporting the physiological teat return, after the process.

### TECHNICAL FIELD OF THE INVENTION

The present invention is related to a system and a method of eliciting milk from all mammals that possess teats, including but not limited to dairy animals and humans.

### BACKGROUND OF THE INVENTION

Most mother mammals, produce milk through a sensitive system which provides the basic nutrients for the growth of their young. In most naturally occurring cases, milk is consumed directly, with the young extracting it directly and according to need (usually several times during a day), from the mother. For an abundance of reasons, nutrition being the main, milk has been extracted from animals since before recorded history. Manual means gave way to a plethora of mechanical means in industrialized communities. Presently, there is a very small variety of principles in the milk extraction equipments used either for animals, or for humans.

The key component and the basis of extracting milk from inside a teat either in nature or in man made efforts is the differential pressure created between the inside of the teat (positive pressure), and the outside of the teat (negative pressure). If the duct(s) that convey(s) the milk outward remains opened, the higher the vacuum is, the faster the milk comes out, up to a peak flow. In an effort to minimize undesirable effects which affect the mammals health, quality and longevity of products and investments and at the same time minimize milk-out time and maximize yields (within the 2-3 milkings that occur per day), pulsed vacuum was introduced in the industry and its application is commonly used and widely accepted. Inspired by mimicking the natural suckling process, a necessary compromise was made between health and efficacy, and it results in the milk and dairy products in the market today.

The limitations presented by contemporary inventions used for extracting milk from mammals, combining pulse and constant vacuum, focus on mammal health, milk-out efficiency and milk quality.

In more detail, mammal health is encumbered or endangered by the following attributes of the prior art, which are designed and constructed for a specific range of occurring teat sizes and are wider in diameter than said teats. Different teat sizes generally imply different blood flow requirements among other, and potentially more specialized treatment for optimal health maintenance and milk-out efficiency. In many, if not in all, of the prior art milking apparatuses for animals, the teat and teat end are forced to come in frequent and periodic contact, fully of partially, with the milking liner shapes and collapse. Such contact and collapse on the teat by said inventions may traumatize and deform the teat, teat end sphincter, teat sinus (or teat cistern in the mammals possessing teat cisterns like most dairy animals), and may result in facilitating mastitis infections, teat sinus stripping, and the further damage of the animal's health.

In the various stages of the milking process, teat dimensions change according to the milk pressure and its requirements for blood flow. Despite the fact that the changes and requirements of the teat are different in the various stages of the process, vacuum inside the liner and mechanical forces exercised from its repetitive collapsing are in opposition with the particular expressions of teat physiology (e.g. liner exercises compressive forces, when the teat physiology expresses a tendency to be expanded due to high milk pressure and vacuum exercises expanding forces when the teat physiology expresses a tendency to be constricted due to low milk pressure in the teat).

Most contemporary prior art inventions operate at high vacuum and pulsation levels, so as to assure the “suspension” of the milking cups on the teat and to decrease milk-out time. Liner collapsing under such application of vacuum and pulsation may expand, extend, damage and compress the teat sinus and teat-end sphincter mainly in an oval or triangular shape, and may bring the milking cup to creep up on the exposed teat, or may cause stripping or overmilking.

Another adverse effect of exercising high vacuum on the teat, is that vacuum causes blood to concentrate on the highly sensitive area of the teat, damaging the animal in several ways or making it more prone to damage or harm. In addition, the fluctuations of vacuum exercised under the teat end, caused by the repetitive collapse and expansion of the milking liner may return milk back through the teat-end sphincter while it is still open, and into the teat sinus. This milk may contain pathogens from the environment or other animals which may still reside on the milking liner from a drop or prior usage, which in turn may further facilitate mastitis infections.

Fluctuations in milking vacuum can promote the transmission of mastitis pathogens among cows and across the milking cluster among quarters (cross-contamination) and increase the incidence of mastitis. These fluctuations can cause a momentary reversal of the vacuum gradient within the milking cluster, forcing air and milk to be drawn up the short milk tube toward the teat end. The resulting aerosol of milk droplets may reach the teat end (teat end impact) with sufficient velocity to penetrate the teat duct (Thompson, P. D. and R. E. Pearson, 1979, 1983).

Reverse pressure gradients (RPG) due to pulsation can exist in the milking cluster under normal milking conditions (Sagi R., R. S. Gates, and N. R. Scott 1983). The RPG caused by a liner slip, occurs during low milk flow or after milk flow has ceased and in addition to teat end impact, the RPG caused by air admission may allow vacuum inside the teat to be temporarily greater than that within the liner, creating the potential for pathogenic bacteria outside the teat to be drawn into the teat canal (Zehr P. D., D. M. Galton, N. R. Scott, and C. S. Czamiecki, 1985).

Overmilking starts when the milk flow to the teat cistern is less than the flow out of the teat canal. Mouthpiece chamber vacuum normally increases during overmilking and fluctuations become larger. If the vacuum in the teat cistern is higher than beneath the teat end for short periods of time, the RPG across the teat canal may give rise to bacterial invasion of the teat cistern. The RPG may occur during pre-milking teat preparation, attachment of the milking unit, milking, and detachment. Overall risks include empty teats (Rasmussen et al., 1994) and overmilking will therefore increase the possibility of bacteria entering the teat.

During milking, the teat end sphincter opens and closes, and its inside may appear pulled out or as an eversion of the teat end. At the end of milking, the muscles in the sphincter are fatigued from the liner collapsing and leave the sphincter open for a period of time after the milking. This exposes the mammary gland to the risk of infections from environmental bacteria (environmental mastitis).

Environmental infections are thought to result when bacteria lacking specific virulence factors penetrate the teat sphincter (Eberhart et al. 1979; Smith et al. 1985). The bulk of these exposures are thought to occur between milkings. Control of these infections is problematic because the cow's teat sphincter remains open after mechanical milking (McDonald, 1975a).

The cumulative time that mechanical forces are applied to the teat-end by the opening and closing liner is an important factor in the development of teat end Hyperkeratosis (HK). Teat-end HK is the result of mechanical forces applied by the opening and closing liner during milking (Woolford and Phillips, 1978; Grindal, 1990).

Collapsing of liners has been mentioned as a potential advantage in favour of liners in studies. Still, during the time the blood flow in the teat is low, the contracting muscles consume the oxygen available in the tissue until muscle relaxation allows resumption of arterial blood flow to the tissue. It has been shown that muscle contraction is therefore, associated with two aspects of tissue oxygen reduction that accompany the extra-cellular fluids circulatory events of teat contractions: a) Temporary obstruction of arterial blood flow, and b) increased oxygen consumption by the contracting muscle fibres (Williams et al. 1978, 1981 Mein and Williams, 1984; Lefcourt 1982a and b; Hamann and Burvenich, 1994).

Most prior art inventions, draw milk in pulses (alternating between pressure and vacuum), considerably encumbering the speed of milk extraction, thus increasing milk-out time and agitating the flow of milk, which might promote lipolysis which reduces milk quality, and causes the depositing of fat on the walls and tubes of the systems. These deposits may especially decrease the service life of elastomer and plastic components of the systems, and encumber sanitation.

All prior art animal milking apparatuses include or comprise of items that come into contact with the teat and are sanitized and reused after usage. The process currently used for the sanitation of the apparatuses, may promote but cannot guarantee the sterility of the items that come in contact with the teat, and after the sanitation process, they may be the carriers of contaminants. In sub-clinical mastitis cases for example (which provide the animal handler with few indications), contaminated liners can potentially contaminate the next teats that are milked with it and finally the extracted milk, a case further facilitated if the teat is already irritated or even slightly damaged. Especially in automated, robotic milking systems, the use of reusable items may cause the uncontrolled deterioration of the structural or functional integrity and stability, especially of the elastomer components, which can cause damage to the teat, to milk quality and equipment.

The health of the mammal milked, directly affects the quality and quantity of produced milk. The more S.C. Counts in milk rise, milk yield decreases in an inversely proportionate manner. The sicker an animal is the less milk it produces. Milk extracted with most prior art inventions, may contain an increased amount of pathogens, which must be removed before the milk is ready for consumption, usually with pasteurization or UHT methods. Milk which contains high bacterial counts also has a shorter shelf life, and temperature tolerance. Additionally, traces of antibiotics, other treatments, or even remaining bacterial spores may eventually find their way into the final products. These conditions may encumber the productive process, against milk hygiene and quality. Milk quality also affects the price of milk, and may hinder producers from achieving premium prices for their milk, which in turn encumbers efficiency.

Efficiency might also be encumbered from the need to assemble and disassemble items of some of the prior art, and the need to count the uses of reusable apparatuses. Additionally, the use of reusable apparatuses that come in contact with the teat and udder demand further man-hours for the process of sanitation, a burden on the environment from the waste-products of sanitation, and additional space and expenditures for the supply of sanitation methods and their application.

Breastpumps are well-known and used from breastfeeding women to remove/express milk for the infants. The basic types of breastpumps are manual, battery-powered, and electric. One of the most important components of a breastpump is the breast shield, which fits directly over the nipple and forms a seal around the areola. A suction force or a vacuum created by a pump draws the nipple into the tunnel of the breast shield.

The pump's suction strength and cycling rate are also important factors. Breastpumps with high suction pressure and cycling rate often cause discomfort and pain, while others with low ones are ineffective and incapable at extracting milk. Prolonged suction can irritate the nipples and cause breast damage.

The high pressure may cause sore nipples and areola, blisters on the nipple, and damage the sensitive breast tissue. The cycles in low cycling pumps tend to be longer and can cause pain since the breast and nipple tissue is suctioned and pulled for too long.

Most of the breastpumps are limited to only one standard breast shield. Subject to the size and shape of a nipple, what seems to fit to one breastfeeding woman may be completely uncomfortable for another.

A small or tightly fitted breast shield can cause friction against the sides of the tunnel with each vacuum movement of the pump and such friction causes pain, leading to a tender or sore nipple. It can also affect breast emptying and lead to problems with milk supply, because the small breast shield squeezes the small ducts inside the nipple that carry the milk out of the breast. This results in an incomplete milk removal and can lead to plugged ducts, mastitis, and problems with low milk volume and breast engorgement. Breast engorgement makes the breast become full, hard, swollen and having difficulty expressing milk or nursing the infant.

A large or loose breast shield, through the suction applied, causes the nipple to be further sucked, elongated and expanded into the tunnel of the breast shield, resulting in a swallowed and injured nipple and more pain for the breastfeeding woman.

Getting used to a breastpump takes too much time and practice for breastfeeding women. Breastpumps are self operated, and their operation can be easily interrupted when the woman feels pain in the breast and nipple area, commonly from the application of suction.

The pain caused by removing/expressing milk from a sore or otherwise damaged nipple, deters breastfeeding women to remove milk from that breast repetitively and worse said condition and nursing an infant from an injured nipple becomes too painful. Such condition may lead a woman to stop breastfeeding the infant.

Nipple shields are artificial nipples worn over the breastfeeding woman's nipple and areola while the infant is nursing. Generally they are made of soft materials, and the infant sucks on the nipple shield, which has holes at the end of its end to allow breast milk to pass through.

A nipple shield is temporarily used when the infant has difficulty latching on to a flat or inverted nipple, or a premature infant has a tiny mouth and a respective difficulty in maintaining proper latch or has reduced or impaired suction force, or an infant who is nipple confused and prefers an artificial nipple rather than a mother's breast.

Studies indicate that infants get less milk during sucking with a shield because they are unable to compress the milk sinuses beneath the areola very well. The use of a nipple shield may be associated with decreased milk transfer, reduced milk supply, increased nipple pain and/or damage, interference with proper latch on, preference by infant to taste or sensation of shield, and problems to wean the infant from the shield.

The normal process of extracting milk from human breasts, especially before and after the infant's teeth begin to appear, may be hazardous for the condition of the nipple, as the infant rubs its irritated gums, or newly formed teeth, on the nipple, biting on it or creating lesions and other irritations.

In view of the aforementioned and other limitations of the prior art, with regard to teat anatomy, physiology and dynamics, blood flow, teat and mammal safety, milk quality and quantity, milk-out speed, and the overall quality and effectiveness of the productive process, a new system and method of eliciting milk are needed to overcome the aforementioned limitations.

### SUMMARY OF THE INVENTION

The principal scope of the invention is to provide a novel system and method for eliciting milk from mammals, which ensure a safer, more effective, more productive and a complete milk elicitation process for the mammals.

Safety is achieved by having optimal teat end health and an optimal teat end sphincter/lactiferous duct(s). This can be achieved by having no contact with the teat end sphincter/lactiferous duct(s) and by maintaining and supporting the teat's natural defense from any pathogen intrusions. Safety is also achieved by teat end strength and health, by the minimization of teat sinus stripping and elimination of overmilking and vacuum passage in the inner areas of the teat.

Safety may be also achieved by ensuring optimal blood flow on the teat that promotes better oxygenation and maintains it in a healthy condition during and after milking. This may be achieved through the use of an elastic system, which supports teat physiology throughout the milk elicitation process.

Safety may be further achieved by supporting the process of gradual return of the teat, after the milk elicitation process, in its physiological condition. This may be achieved by the contracting forces of the elastic system, which operate regressively during system's removal.

Efficiency is achieved by ensuring optimal mammal and teat health, having a complete milk elicitation process, maximal and continuous milk flow, least milk elicitation time and least time of system attachment on the teat. Efficiency may be also achieved by optimal milk flow management, in continuous equilibrium with the vacuum, atmospheric air, intra-mammary pressure and desired teat blood flow.

Efficiency may be further achieved by the easy handling of the milk elicitation system, by significantly simplifying the configuration of the system, reducing the number of components of the system, reducing the cost of manufacturing the system, reducing the cost and time of sanitation processes.

Yet another scope of the invention is to provide a new system and method for eliciting milk from mammals, which exercises vacuum only on the area around the teat end sphincter/lactiferous duct(s).

Another scope of the invention is to provide a new system and method for eliciting milk from mammals, which protect the teat, teat end and milk flow from contacting contaminants. This may be achieved by optimal adaptation and attachment of the system on the teat throughout the process.

Another scope of the invention is to provide a new system for eliciting milk from mammals, which eliminates the possibility of potential transfer of pathogens from the surface of one teat on the surface of the other. This may be achieved by the application of a single-usage elastic system.

Another scope of the invention is to provide a new milk elicitation system and method for human breasts, which provide protection to the areola and nipple and optimal breast emptying, effectively encouraging milk flow, maintaining and improving the milk supply in the breast, assisting the infant to latch on properly to the breast, by further making milk elicitation a healthy, comfortable and satisfactory process for the breastfeeding mother and the infant.

Another scope of the invention is to provide a new milk elicitation system and method on human breast, which provide the breastfeeding mother to keep on nursing the infant for a longer time, and therefore he/she will continue to have available the benefits of his/her mother's milk.

A milk elicitation system for mammals according to the invention may be comprised of an elastic system and a vacuum supply. Through the application of vacuum via the vacuum supply, the elastic system forms, differentiates, seals and adapts to a respective teat, from the area the teat ends (around the area of exit of the lactiferous duct) until up to where the udder ends and the teat begins, without coming in contact with the sphincter/lactiferous duct(s) at the teat-end. The elastic system interacts proportionately with an area up to the entire teat skin surface and according to tendencies expressed by teat physiology and teat dynamics according to the milk pressure change, blood flow, and the effects of applied vacuum, throughout the milk elicitation process, and supporting the physiological teat return, after the process.

A method of milk elicitation from mammals according to the invention may include a system comprised of an elastic system and a vacuum supply. Through the application of vacuum via the vacuum supply, the elastic system forms, differentiates, seals and adapts to respective teat, from the area the teat ends (around the area of exit of the lactiferous duct) until up to where the udder ends and the teat begins, without coming in contact with the sphincter/lactiferous duct(s) at the teat-end. The elastic system interacts proportionately with an area up to the entire teat skin surface and according to tendencies expressed by teat physiology and teat dynamics according to the milk pressure change, blood flow, and the effects of applied vacuum, throughout the milk elicitation process, resulting in continuous and complete milk elicitation and supporting the physiological teat return, after the process.

These together with other scopes, features and advantages that characterize the invention are achieved by a system and a method having the main features as defined in the respective claims and will become evident upon examination of the ensuing detailed description of the preferred embodiments where reference numbers respond to respective items throughout.

### BRIEF DESCRIPTION OF THE DRAWINGS

FIG. 1 presents a perspective view of an embodiment of an elastic system.

FIG. 2 presents a vertical section of an embodiment of a milk elicitation system, in one application of the claimed method, with the elastic system connected to a vacuum supply, before it is formed, differentiated and adapted on the cow teat.

FIG. 3, presents a vertical section of an embodiment of a milk elicitation system, in one application of the claimed method, with the elastic system connected to a vacuum supply, during its progressive formation and differentiation on the cow teat, when the milk pressure in the teat is increasing.

FIG. 4 presents a vertical section of an embodiment of a milk elicitation system, in one application of the claimed method, with the elastic system connected to a vacuum supply, after the elastic system is formed, differentiated, sealed and adapted to the cow teat and during operation when the milk pressure in the teat is at its peak.

FIG. 5 presents a vertical section of an embodiment of a milk elicitation system, in one application of the claimed method, with the elastic system connected to a vacuum supply, at the end of the milk elicitation process when the milk pressure in the cow teat is decreasing.

FIG. 6 presents a vertical section of an embodiment of a milk elicitation system, in one application of the claimed method, with the elastic system connected to a vacuum supply, at the end of the milk elicitation process when the vacuum is switched off and the elastic system regressively contracts and is removed from the cow teat supporting its return in the physiological condition.

FIG. 7 presents a view of a vertical section of an embodiment of a milk elicitation system, in one application of the claimed method, with the elastic system connected to a vacuum supply, before it is formed, differentiated and adapted on the human breast.

FIG. 8 presents a vertical section of an embodiment of a milk elicitation system, in one application of the claimed method, with the elastic system connected to a vacuum supply, after the elastic system is formed, differentiated and adapted to the breast and during operation.

FIG. 9 presents a vertical section of an embodiment of a milk elicitation system, in one application of the claimed method, at the end of the process when it is removed from the breast.

### DETAILED DESCRIPTION OF PREFERRED EMBODIMENTS

The shown embodiments according to the invention manifest milk elicitation from mammals, through the use of a system (500) for each teat, which may be comprised of an elastic system (100), and a vacuum supply (200).

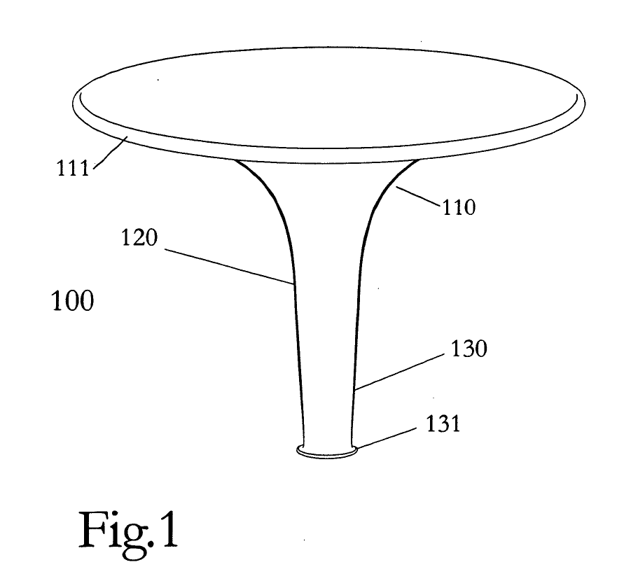

According to FIG. 1, the elastic system (100) is depicted in a roughly cone shaped structure.

The elastic system's (100) main characteristic are its elastic properties, which allow it to perform its basic functions. These include formation, differentiation, sealing and adaptation on the full dynamic spectrum of different teat sizes (static and dynamic lengths and widths) for which each one is designed, as well as contraction of the elastic system (100) upon its removal.

The length, width and wall thicknesses of the elastic system (100) are determined according to the teat anatomy, length and width of the mammal in consideration, in static and dynamic conditions.

The upper area (110) of elastic system (100), may fashion at least one area (111) around the top which displays less elasticity and interacts more forcefully with deforming forces. This may be due to construction characteristics (more material thickness) or due to material choices (more rigid material(s) in said area). Said area (111) may be annular or of any suitable shape to provide for the application of the method.

The mid area (120) of elastic system (100) follows up the downward conical curve of upper area (110).

The lower part of mid area (120) follows up in a near vertical curve resulting in lower area (130). The lower area (130), may fashion at least a lower end area (131) which may also display less elasticity and may interact more forcefully with deforming forces. Same as the upper annular area (111) this could be due to construction characteristics (more material thickness) or due to material choices (more rigid material in said area) and said area (131) may be annular or of any suitable shape to provide for the application of the method.

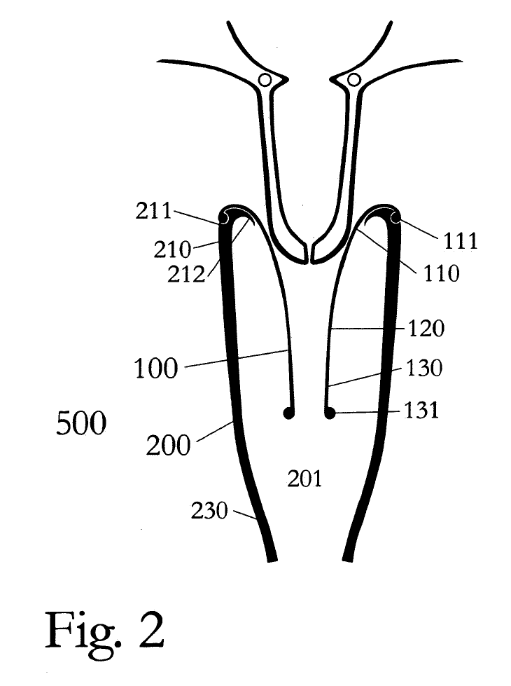

As shown in FIG. 2, annular area (111) serves as an association means of the elastic system (100) to vacuum supply (200). Annular area's (111) section surface may be circular, square or a combination of surfaces that provide adequate attachment of elastic system (100) to vacuum supply (200) during operation and the application of vacuum, and sufficient sealing of the system (500). Said annular area (111), may be a seamless continuation of elastic system (100) and association with the vacuum supply (200) could occur in any means suitable for the application of the method.

The lower end annular area (131) may serve to seal or maintain pressure communication and equilibrium between the inside of elastic system (100) and the inside of vacuum supply (201) and to restrain the teat inside elastic system (100) in the case the teat length reaches the elastic system's (100) length. To provide an adequate application of the method, the lower end annular area's (131) vertical section surface may be circular, square, a combination of surfaces or an extension into a contact point with the vacuum supply (200) or directly to a milk collection receptacle. In specific applications of the elastic system (100) on long teats (like buffalos'), an extension may be fashioned in the annular area (131) of the elastic system (100), which may be fixed at least on one area of the vacuum supply (200) to assist in elastic system's (100) removal practices from the teat (not shown).

Vacuum supply (200) may be comprised of a rigid, concave element, the inner area (201) of which transfers vacuum, milk and atmospheric air.

The vacuum supply (200) may have any shape which supports the effectiveness and efficacy of its use (especially its milk and vacuum carrying properties), with preferred shape that in which its lower area (230) may narrow gradually as compared to its upper area (210). Said structure as well as similar structures may be more preferable since they offer smoother flows of milk from the teat and minimize turbulent flows which promote prevention of milk lipolysis.

The vacuum supply (200) may fashion at least one attachment fitting area (211) for the attachment of the elastic system's (100) top annular area (111).

Fitting area (211) may be circumferential and may be located in a variety of internal and external locations on the vacuum supply (200), according to particular embodiments, according to particular characteristics of the general class and respective teat/udder structure of the mammals being milked, according to elastic system (100) replacement practices, according to manual or automated installation/removal practices and according to preferred construction methods.

At least one circumferential rim (212) may exist in the interior or external area of the vacuum supply (210). Said rim (212) may be circumferential, and may be fashioned in various possible configurations along the upper area (210) of vacuum supply (200) or in the fitting area (211) of the vacuum supply, including but not limited to a number of vertically alternating rims (not shown), and/or a number of repeating rims with radial symmetry according to the vertical axis. The rim (212) may extend so that its continuation may form an elastic system (100), as if the vacuum supply (200) and elastic system (100) are constructed as a single item (not shown).

The vacuum supply's upper end (210), fitting area (211) and rim (212) may be structured such that an interface support area is provided to the venous plexus for the animals that possess one.

As shown in FIG. 2, the mid (120) and lower (130) cross-section diameters of the elastic system (100), before it is formed, differentiated and adapted on the teat, are smaller than the diameter of the teat end (the area around the teat end sphincter). Said structure is more suitable for different teat diameters of the same mammal category. For example, for a cow teat with large diameter, the elastic system (100) will be sealed from atmospheric air influx at the mid area (120). In the case of a cow teat with small diameter, the elastic system (100) will be sealed from atmospheric air influx at the end of mid area (120) or at lower area (130). The cow's teat is shown after the needed pre-stimulation, in accordance with the principles widely known and accepted in the art. The accumulation of the alveolar milk in the udder and teat cistern instigates an increase in internal milk pressure and has caused a circumferential widening and an elongation of the teat and the teat cistern, and Fürstenberg rosette and streak canal are (partially or fully) dilated. When the system (500), in a manual or automated manner, approaches the teat end, vacuum is supplied to the inside area of vacuum supply (201) through its lower end area (230).

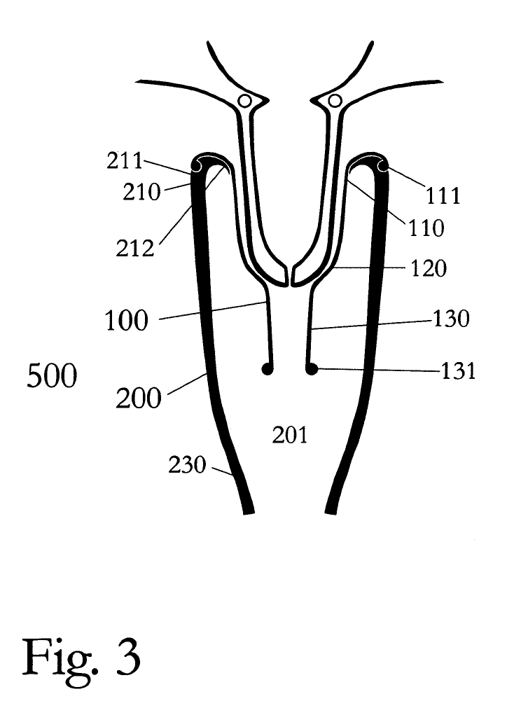

As show in FIG. 3, the elastic system, with the application of vacuum, begins to progressively form and differentiate on a teat through its upper end area (110) and the mid area (120), in accordance with the increase of vacuum at the teat end and the milk pressure in the teat cistern and as to the length and circumference of the teat from the area around the teat sphincter and up to the whole teat.

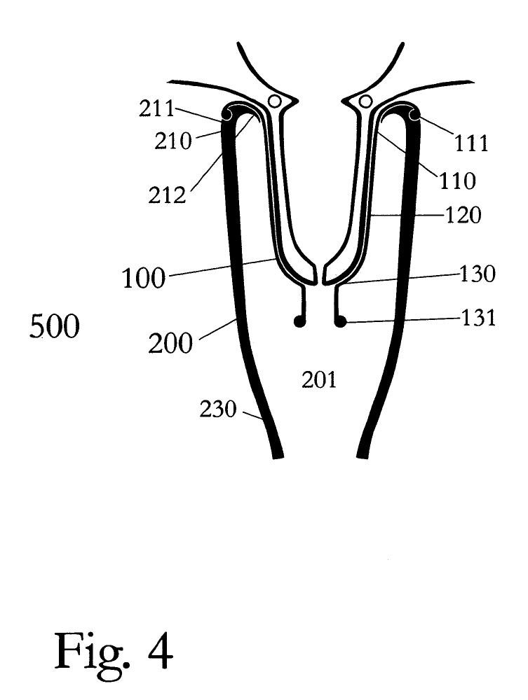

In FIG. 4, the elastic system (100) is fully formed, differentiated, sealed from atmospheric air influx at least at the elastic system's lower area (130) at the teat end, around the teat end sphincter and adapted on the teat from the area the teat ends (around the area of exit of the lactiferous duct) until the area where the udder ends and the teat begins, without coming in contact with the teat-end sphincter.

The internal pressure exercised by milk in the teat cistern and teat end streak canal and the vacuum exercised at the teat end sphincter area, overcome the pressure forces exercised by the elastic system (100) around the area of the teat end sphincter, and teat sphincter opens and continuous milk elicitation flow occurs.

The admission of atmospheric air, related to milk flow transportation, may be provided in a controlled, constant or periodic manner (not shown) from any area of the vacuum supply (200), or any point or area or element in the ensuing milk transportation line.

As shown in FIG. 4, the teat dynamics are presented in the stage where the continuous milk flow has reached its peak. The elastic system (100) responds interactively to the dynamics of the teat, and contains it to a shape promoting optimal health and productivity characteristics. The forces exerted on the teat by the elastic system (100) are interactive and proportionate with the forces applied by the muscle structure of the teat, maintaining the teat shape. The forces applied by the elastic system (100) on the periphery of the teat, have radial directions towards the vertical axis of symmetry of the teat. In the same manner as the horizontal expansion of the teat, the vertical forces exerted by the elastic system (100) on the teat push the teat upwards, and significantly avert the undesirable teat lengthening; apart from stressing the vertical muscles less, also promoting better blood flow to the teat end. The elastic system (100) achieves the maintenance of the teat in the shape most fitting to optimal milk flow, optimal blood flow for the supply of teat with adequate blood for its healthy function.

In simpler terms, the whole teat is reinforced by the pressure of the elastic system (100) forces, which act as an external set of the same muscles, congruently interacting with the teat's muscle structure. In other words, the forces exerted by the elastic system (100) on the teat, are directly proportional to the tendencies expressed by the teat physiology. The larger a tendency for elongation, the more forceful the response from the elastic system (100) to contain the teat to an optimal length. Likewise, the larger the tendency for circumferential widening, the more forceful the response of the elastic system (100) for containment of same.

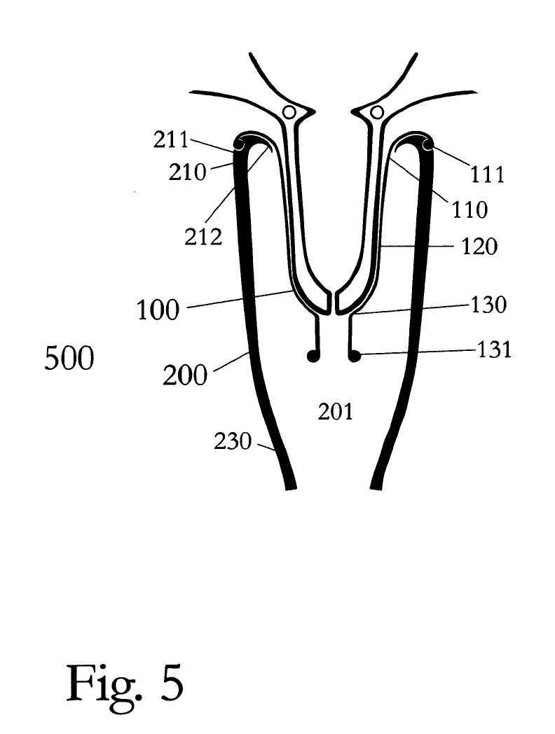

As shown in FIG. 5, during the end of the milk elicitation process, the internal teat pressure progressively falls as a result of less milk in udder and teat. The elastic system (100) adjusts on the teat according to its needs at this stage. The decrease of pressure in the teat causes the relaxation of the horizontal and vertical muscle structure of the teat walls and an accompanying decrease of pressure from forces of the elastic system (100). As portrayed, the pressure exerted by the elastic system (100) also interacts less with the teat end area The gradual decrease of the milk flow is accompanied with gradual contraction of the teat canal and teat end sphincter and the elastic system (100) responds to this decrease of pressure, up to a complete milk-out, eliminating stripping, overmilking and the passage of vacuum towards the inner areas of the teat or udder. As there is always a tendency of the sphincter to remain closed, there is always a congruent force exerted by the system on the teat end. This force decreases as milk flow drops to zero, but is still in effect to close the sphincter from intrusion of vacuum in the teat cistern.

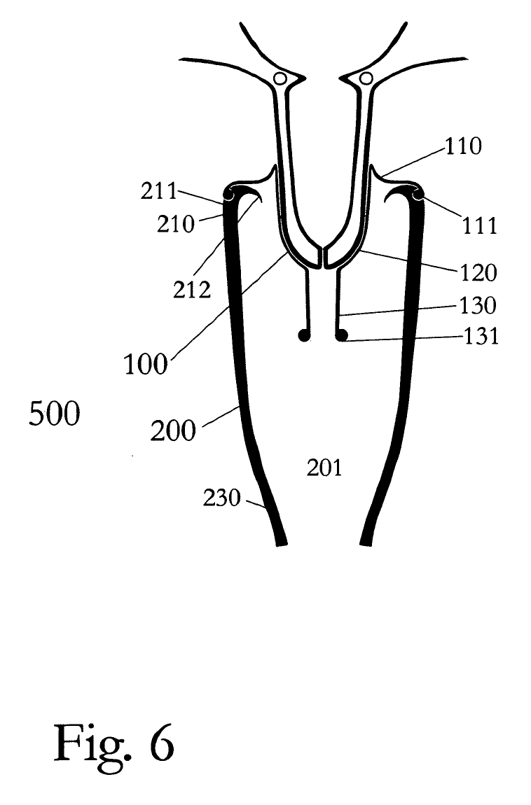

As shown in FIG. 6, by switching off the vacuum in the system (500) at the end of the elicitation process, the elastic system (100), in absence of the expanding forces of vacuum, regressively and gradually contracts and returns to its original shape starting from the elastic system's (100) lower area (130), mid area (120) and upper area (110). The elastic system (100) exercises contracting forces vertically and radially around the teat end sphincter/lactiferous duct(s) and towards the central axis, unsealing and disengaging gradually from the teat, without coming in contact with the sphincter/lactiferous duct(s) at the teat-end, supporting the teat return in its physiological condition. System (500) removal can be done in a manual or automated manner in accordance with the preferred applications.

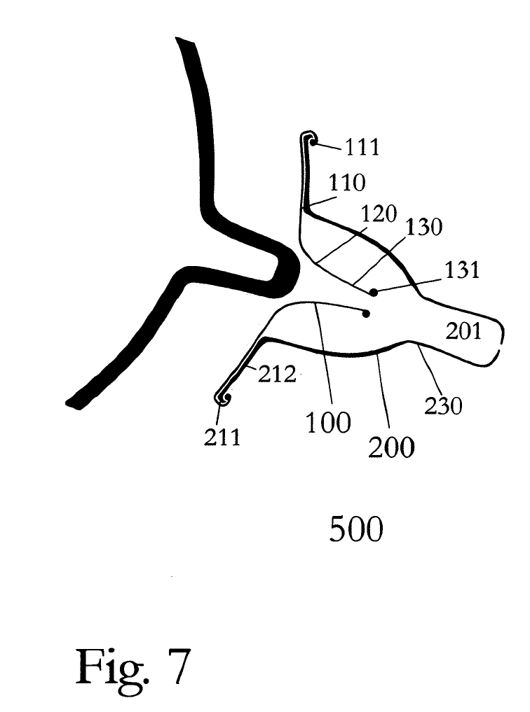

The embodiment depicted in FIG. 7, is particularly focused on eliciting milk from a human breast. The elastic system (100) is shown associated with the vacuum supply (200) at the upper area (111). The elastic system's (100) mid and lower diameter before it is formed, differentiated and adapted on the breast, is smaller than the diameter of the nipple end.

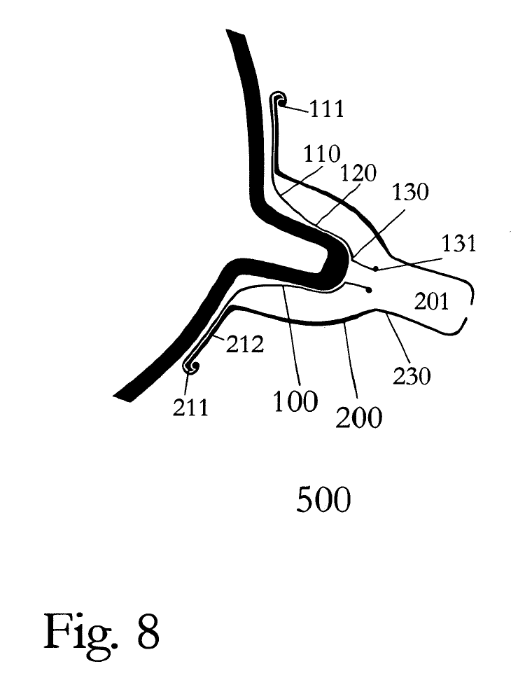

According to FIG. 8, with the application of vacuum (constant/periodic) through the vacuum supply (200), which can be placed in the infant's mouth, or any kind of breast pump, the elastic system (100) forms, differentiates, seals and adapts to the shape, anatomy and physiology of the breastfeeding mother's breast, around the areolar tissue and surrounding skin of the breast, without coming in contact with the nipple ducts at the nipple end area.

The vacuum effects, exercised from the infant's mouth (the effect of indirect suckling) or a breast pump, are mainly applied on the area of nipple end, around the nipple ducts, and milk is elicited freely and safely through them.

The elastic system (100) interacts with the breast and nipple of the breastfeeding mother as to the dynamics of the milk pressure in the ducts during milk elicitation, and the milk flows out of the nipple ducts easily, quickly and without any pain.

When the infant suckles on an appropriately shaped vacuum supply (200), he/she supplies the vacuum necessary for the operation of the system (500), and consumes the milk directly.

Even when the infant might bite or otherwise damage the nipple, he/she has no direct contact with it. The only contact that the infant's mouth has is the vacuum supply, which can be designed to a similar shape of the breastfeeding mother nipple.

By eliciting the milk, with the system (500) placed on a vacuum pump, the milk can become available for consumption from the infant at a later time.

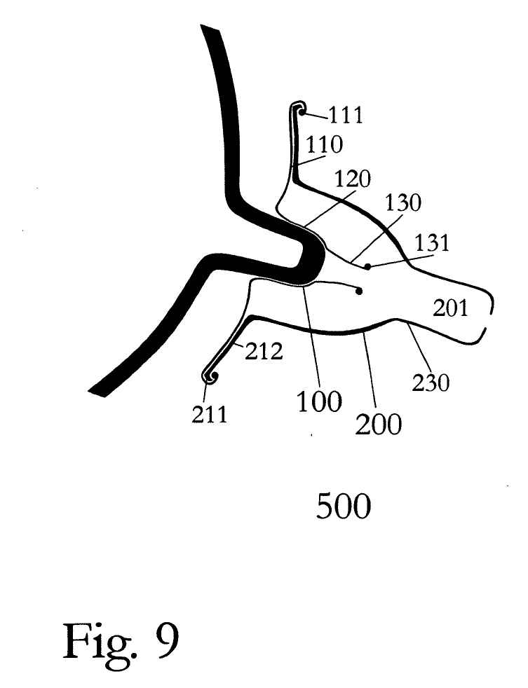

As shown in FIG. 9, after the milk elicitation, the system (500) removal supports the process of return to the breast in its physiological condition.

According to the embodiments portrayed, the present invention ensures a dynamic equilibrium between the variables of milk elicitation speed (i.e. continuous milk flow magnitude), applied vacuum, atmospheric air, change of the milk pressure in the teat and teat blood flow. This is achieved through the forces exerted on the teat skin by the elastic system (100) according to the teat dynamics throughout the milk elicitation process.

The fact that the forces exerted by the elastic system (100) on the teat are interactive and directly proportionate to the tendencies expressed by teat physiology and the teat muscle structure, ensure that there are never sores on the teat skin. Healthy teat skin increases the teat's resistance to intrusions of pathogens, during and after milk elicitation.

The milk flow magnitude during the milk elicitation process is a variable subject to following parameters: quantity of available/cisternal and alveolar milk, the pressure created from milk in udder and teat, and the vacuum exercised at the teat end sphincter/lactiferous duct(s) to elicit this milk. Opening and closing of the teat end sphincter/lactiferous duct(s) is always subject to the change of these parameters.

The dynamics of the elastic system (100) are in accordance with teat needs throughout the milk elicitation process, providing favorable conditions and sensations, which promote optimal blood flow and oxygenation of the teat tissue. Said sensations are related to the promotion of timely oxytocin release, leading to greater quantities of alveolar milk becoming available as cisternal milk for elicitation, further leading to increased productivity and capacity for comprehensive parameterization according to specific physiological mammal requirements.

The system's (500) embodiments applying the method are available through the differentiation of a number of the variables and options described hitherto. They mainly include the association of the elastic system (100) with the vacuum supply (200), the formation, differentiation, sealing at least around the area of the teat end sphincter/lactiferous duct(s) and adaptation of the elastic system (100) on the teat, the proportionate interaction of the elastic system (100) with the teat dynamics and the blood flow and the tendencies expressed by physiology of same, and the administration of continuous milk flow and atmospheric air in the vacuum supply (200) and system's (500) removal practices from the teat. The method remains the same regardless of embodiment; continuous and complete milk elicitation while supporting the teat via the use of a elastic system (100) that is formed, differentiated, seals at least around the area of the teat end sphincter/lactiferous duct(s) and is adapted on teat, while the elastic system (100) interacts proportionately with the teat dynamics and the blood flow and the tendencies expressed by physiology of same, while administrating milk flow and atmospheric air throughout the milk elicitation process, while the system's (500) removal supports the teat's return to its physiological condition. The functionality, efficiency and practicality of embodiments are only according to specific desired effects that need to be attained, especially with regards to specific mammal anatomy/physiology.

Given mean teat size dimensions, teat muscle structure and tolerances to vacuum for each individual mammal class to be milked, the elastic system (100) may be constructed with a very large variety of wall thicknesses (which can modify elasticity and increase or decrease applied forces for a given material, locally or totally, in static or dynamic conditions), lengths, curves or shapes. Milk elicitation performance parameters thus rely only on conformation and adaptation of the elastic system (100) to the mammals being milked, and the vacuum that elicits said milk.

Some embodiments may fashion the elastic system (100) and the vacuum supply (200) molded or otherwise constructed from a single or multiple materials, as a single element, or any number of assembled elements. Embodiments are presented as an assembly of elements, their individual constituents being more simple and cost effective to produce.

The elastic system (100) and vacuum supply (200) may be constructed of any materials displaying the properties of elasticity, flexibility and/or rigidity explained above. These materials may be homogenous, or a combination of multiple materials, opaque, transparent or translucent according to need. These materials may additionally display a variety of bacterial tolerances or antibacterial properties, radiation tolerances and/or chemical tolerances, and may be recyclable.

The vacuum supply (200) may be reusable, its service life depending on material choices, handling, hygiene and sanitation processes.

The elastic system (100) may be constructed with a variety of service lives; for a single use or a limited number of uses. A significant advantage of using a single-use elastic system (100), is that there is no need for sanitation after its use and that there is no possibility that pathogens might be potentially transferred from the surface of one teat on the surface of the other, potentially infecting several teats of the same animal, or different members of a herd.

The system (500) of milk elicitation from mammals may be constructed as a single item, and it may use an additional single-use elastic system (100) (resembling a membrane), which can be removed or allowed to temporarily stay on teat after the elicitation process, to protect the teat from potential environmental infections.

It is noted that throughout the text, the characterizations of elastic and rigid, are used to denote the properties of the system (500) and its components during vacuum application, and do not limit the material choices from which the system and its components are fashioned from. It is considered obvious to those knowledgeable in the art, that more complicated structures or combinations of lines or curves might produce similar performance results; this solution is presented as the simplest functional, applicable, efficient and easiest and more financially feasible to be manufactured, even in very small numbers or diffuse locations.

The above referenced embodiments are only illustrative and the present invention is limited only by the claims that follow.

1. A system (500) for eliciting milk from mammals comprises:
an elastic system (100), said elastic system (100) is designed in a roughly conical shaped structure with mid (120) and lower (130) areas having diameters smaller than the diameter of the teat end, said elastic system comprises an upper area (110) which includes at least one association means (111), which serves as an association element of the elastic system (100) to vacuum supply (200); a mid area (120) which interconnects with said upper area (110); a lower area (130) which interconnects with said mid area (120) and includes at least one restraining means (131), which provides restraining the teat inside the elastic system (100) and maintains pressure communication and equalization between the inside of elastic system (100) and the inside (201) of vacuum supply (200);
a vacuum supply (200) for holding an elastic system (100) in its inner area (201), for application of vacuum and for transfer of milk flow, said vacuum supply (200) comprises an upper end area (210), which includes at least one attachment fitting area (211), and which serves for the attachment of a elastic system's (100) association means (111); and at least one rim fashioned circumferentially (212).

2. (canceled)

3. The system (500) for eliciting milk from mammals according to claim 1, wherein the association means (111) of the upper area (110) and the restraining means (131) of the elastic system (100) have an annular shape.

4. The system (500) for eliciting milk from mammals according to claim 1, wherein the association means (111) of the upper area (110) of the elastic system (100) is of a section surface with suitable shape that provides adequate attachment of elastic system (100) to vacuum supply (200) during operation and the application of vacuum.

5. The system (500) for eliciting milk from mammals according to claim 1, wherein the restraining means (131) of the lower area (130) of the elastic system (100) comes to a contact point with the vacuum supply (200) to assist in elastic system's (100) removal practices from the teat.

6. The system (500) for eliciting milk from mammals according to claim 1, wherein the restraining means (131) of the lower area (130) of the elastic system (100) ends to a milk collection receptacle.

7. (canceled)

8. The system (500) for eliciting milk from mammals according to claim 1, wherein the vacuum supply (200) is the milk collection receptacle.

9. The system (500) for eliciting milk from mammals according to claim 1, wherein vacuum is provided constantly to the inside area (201) of the vacuum supply (200).

10. The system (500) for eliciting milk from mammals according to claim 1, wherein vacuum is provided periodically to the inside area (201) of the vacuum supply (200) by an infant's mouth.

11. The system (500) for eliciting milk from mammals according to claim 1, wherein the fitting area (211) is fashioned circumferentially for attachment to an association means (111), dependent on teat/udder structure, replacement practices for the elastic system (100), manual or automated replacement and preferred construction methods.

12. (canceled)

13. The system (500) for eliciting milk from mammals according to claim 1, wherein the vacuum supply upper end (210), fitting area (211) and rim (212) are designed to provide an interface support area to the teat venous plexus.

14. (canceled)

15. (canceled)

16. The system (500) for eliciting milk from mammals according to claim 1, wherein the elastic system (100) and vacuum supply means are constructed for a variety of service lifetimes.

17. The system (500) for eliciting milk from mammals according to claim 1, wherein the elastic system (100) is single-usage.

18. The system (500) for eliciting milk from mammals according to claim 1, wherein the elastic system (100) and vacuum supply (200) are constructed as a single element.

19. A method of eliciting milk from mammals, with the use of a system (500) as claimed in the above claims, the method comprising the steps of:
providing an elastic system (100), which with the vacuum application through vacuum supply (200) progressively forms, differentiates, seals at least at the teat end, around the area of sphincter/lactiferous duct(s) and adapts to a respective teat from the area the teat ends (around the area of exit of the lactiferous duct) until up to where the udder ends and the teat begins, without coming in contact with the sphincter/lactiferous duct(s) at the teat-end;
providing forces exercised by the elastic system (100) around the area of the teat end sphincter are overcome by the internal pressure exercised by milk in the teat cistern and teat end streak canal and the vacuum exercised at the teat end sphincter area, open the teat end sphincter/lactiferous duct(s);
providing the elastic system (100) interacts proportionately with an area up to the entire teat skin surface according to the tendencies expressed by teat physiology and dynamics, while the forces exerted on the teat by the elastic system (100) throughout the milk elicitation process, ensure a dynamic equilibrium between the variables of applied vacuum, atmospheric air, change of the milk pressure and blood flow in the teat and milk elicitation speed; and
providing the elastic system (100), by switching off the vacuum, unseals, regressively contracts and exercises contracting forces vertically and radially around the teat end sphincter/lactiferous duct(s) and towards the central axis, disengaging gradually from the teat, without coming in contact with the sphincter/lactiferous duct(s) at the teat-end.
